SEQUENCE LISTING (2 sequences)

SEQ ID NO: 1 - protein, 5 aa, Homo sp.
YGFGG

SEQ ID NO: 2 - protein, 14 aa, Homo sp.
ALKRQGRTLYGFGG

(19) United States
(12) Patent Application Publication
(10) Pub. No.: US 2025/0213644 A1
(43) Pub. Date: Jul. 3, 2025

(54) COMPOSITIONS AND METHODS FOR TREATMENT OF BONE-RELATED DISEASE OR DISORDER

(71) Applicant: Yankel GABET, Jerusalem (IL)

(72) Inventors: Yankel GABET, Jerusalem (IL); Bitya RAPHAEL-MIZRAHI, Mevaseret Zion (IL); Michal EGER, Be'er Ya'akov (IL)

(73) Assignee: Yankel GABET, Jerusalem (IL)

(21) Appl. No.: 18/685,806

(22) PCT Filed: Aug. 22, 2022
(86) PCT No.: PCT/IL2022/050915

§ 371 Date: Feb. 22, 2024

Related U.S. Application Data
(60) Provisional application No. 63/235,816, filed on Aug. 23, 2021.
(60) Provisional application No. 63/299,019, filed on Jan. 13, 2022.

(51) Int. Cl. A61K 38/08; A61K 31/00; A61K 38/10; A61P 19/00

(52) U.S. Cl. CPC A61K 38/08; A61K 31/658; A61K 38/10; A61P 19/00

(57) ABSTRACT

The present invention provides pharmaceutical compositions and methods for use in accelerating bone repair and growth and attenuating inflammation-induced osteolysis. In particular, the present invention provides pharmaceutical compositions comprising a peptide comprising the sequence YGFGG for use in treating osteolysis. The present invention further provides pharmaceutical compositions comprising a peptide comprising the sequence YGFGG and CBD for use in accelerating bone repair and/or bone growth in a subject having a bone fracture.

### CROSS-REFERENCE TO RELATED APPLICATIONS

This application is a U.S. Nationalization of PCT International Application No. PCT/IL2022/050915 filed Aug. 22, 2022, which claims benefit to U.S. Provisional Application No. 63/235,816 filed Aug. 23, 2021 and U.S. Provisional Application No. 63/299,0191 filed Jan. 13, 2022, the disclosure of each of which is hereby incorporated by reference in its entirety.

### FIELD OF THE INVENTION

The present invention relates to compositions and methods for treating bone-related disease or disorder. In particular, the compositions of the present invention comprise a peptide comprising an active form of osteogenic growth peptide (OGP), and are useful in treating inflammation-induced osteolysis. The present invention further relates to compositions comprising an active form of OGP and Cannabidiol (CBD) for use in acceleration of bone formation and regeneration, bone growth and bone fracture repair.

### SEQUENCE LISTING

The instant application contains a Sequence Listing which has been submitted electronically in .xml format and is hereby incorporated by reference in its entirety. Said sequence listing file, created on 1 Aug. 2022, is named RAMOT096 PCT-Seq.xml and is 2,669 bytes in size.

### BACKGROUND

A bone fracture is a medical condition in which there is a break in the continuity of the bone. A bone fracture can be the result of high force impact or trivial injury. The fracture may be a result of certain medical conditions that weaken the bones, such as osteoporosis or cancer-affected bone. Bones can also fracture as a result of repeated small stresses or strains. This type of fracture is known as a fatigue fracture and is common in athletes. Bone fractures are highly prevalent, and although most fractures in young and adult patients heal correctly, 5-10% of these fractures suffer from delayed or impaired healing. Treatments of at-risk fractures include invasive procedures that are expensive, time-consuming, and associated with morbidity, as well as non-invasive therapies, whose effectiveness is questionable. There is a growing need for new therapeutic approaches that are effective in enhancing fracture healing, inexpensive and have tolerable side effects.

A bone fracture may be performed intentionally, for example by a bone dissection during an orthopedic surgery. Many different types of surgeries include bone dissection. For example, osteoclasis is an intentional surgical fracture of bone performed to correct deformity, e.g. to straighten a bone that has healed crookedly following a fracture. Osteotomy is a surgical procedure in which a bone is cut to shorten, lengthen, or change its alignment. It is performed to correct musculoskeletal deformities, such as coxa vara, genu valgum, and genu varum, as well as dentofacial deformities. Osteotomy is also a part of a distraction osteogenesis surgery, which involves bone dissection, distraction and a further consolidation of the elongated bone.

Fracture repair involves a coordinated and complex processes of cell and tissue proliferation and differentiation. Many players are involved, including growth factors, inflammatory cytokines, antioxidants, bone breakdown (osteoclast) cells and bone building (osteoblast) cells, hormones, amino acids, and numerous minerals and other nutrients.

Osteolysis is a progressive condition where bone tissue is destroyed due to resorption of bone matrix by osteoclasts. In this process, bone tissue is degraded, often irreversibly. Osteolysis may be caused by variety of pathologies like bone tumors, cysts, or chronic inflammation, and may be caused by the vicinity of implants or prosthesis.

Periodontal-related diseases affect the tissues that surround and support the tooth. Periodontitis is an inflammatory lesion that is accompanied by soft tissue loss of integrity (attachment to the tooth) and bone resorption in the tooth-supporting structures. It initiates by a biofilm that forms on the tooth surface and induces an inflammatory response in connective tissue leading to the stimulation of osteoclasts and periodontal bone loss. In 2009-2012, 46% of US adults had periodontitis, with 8.9% having severe periodontitis. Periodontitis prevalence was positively associated with increasing age and was higher among males (Eke et al, 2015, J Periodontol. 2015 May; 86(5): 611-622).

Peri-implant infectious diseases commonly include peri-implant mucositis which is restricted to the peri-implant mucosa and peri-implantitis which also affects implant-supporting bone. While the former is known to be reversible upon conventional treatment including utilization of different manual ablations, laser-supported systems as well as photodynamic therapy, which may be extended by local or systemic antibiotics, the latter is very difficult to treat. Most treatments aim at reducing the number of bacteria on the implant and to stop, or at least attenuate the rate of bone loss. However, these treatments rarely restore what was lost and surgical therapies to regain osseointegration (a direct connection between living bone and the surface of a load-bearing artificial implant) are very expensive with a very low success rate. Currently, peri-implant infective diseases remain one of the most frequent complications affecting both the soft and hard tissues surrounding an implant that can even lead to implant loss (Smeets et al. Head & Face Med. 2014, 10:34). In addition, the increasing use of titanium implants in dentistry is associated with an increased concern regarding inflammation-induced osteolysis due to small titanium particles around implants. These particles originate from mechanical scrapping of the Ti implants or chemical erosion and corrosion. In particular, biomolecules including lipopolysaccharide (LPS), a component of Gram-negative bacterial cell walls and cause of inflammation have been shown to interact strongly with Titanium (Ti) and modify its corrosion resistance. Gram-negative microbes are abundant in biofilms which form on dental implants.

Osteogenic Growth Peptide (OGP) is a 14 amino acid C-terminal peptide encoded by an alternative translation initiation codon along the mRNA for Histone H4. The 14-amino acid peptide is proteolytically cleaved to generate its active pentapeptide form H4(99-103) (OGP(10-14)); also denoted herein 5aa-OGP). This 5aa-OGP pentapeptide stimulates the proliferation and differentiation of osteoblasts (the bone forming cells) in vitro and stimulates bone formation in vivo. OGP was reported to enhance fracture healing in rats by stimulating osteoblasts and bone formation, leading to increased volume and density of the mineralized callus (Gabet et al. Bone 35 (2004) 65-73).

U.S. Pat. No. 6,479,460 discloses compositions comprising synthetic pseudopeptide derivatives of osteogenic growth peptide (OGP) and OGP(10-14), and their use in stimulating the formation of osteoblastic or fibroblastic cells, enhancing bone formation in osteopenic pathological conditions, repairing fractures, healing wounds, grafting of intraosseous implants, reversing bone loss in osteoporosis and other conditions requiring enhanced bone cells formation.

The endocannabinoid system (ECS) and its roles in health and disease are the focus of increasing interest, and its components have been considered as promising targets for a wide range of diseases. Cannabidiol (CBD) is the major non-psychotropic phytocannabinoid compound present in the plant Cannabis sativa, making up to 40% of the cannabinoids in Cannabis extracts. CBD therapeutic activity in bone repair and regeneration has been more recently studied (Kogan et al. J Bone Min Res 2015). CBD enhanced the quality and biomechanical properties (e.g. bone strength) of the newly formed bone via the stimulation of collagen cross-linking and maturation. This effect was statistically significant in newly formed bone after 6 to 8 weeks of treatment in rats.

There still remains an unmet need for safe and efficient pharmaceutical compositions and methods for treating and preventing osteolysis, fracture healing and other bone-related diseases and disorders.

### SUMMARY OF THE INVENTION

The present invention provides pharmaceutical compositions and methods for treating bone-related diseases or disorders. The present invention provides in some embodiments pharmaceutical compositions comprising a peptide comprising the active form of osteogenic growth peptide (OGP), and methods for treating osteolysis. The compositions of the present invention may beneficially be used for inhibiting osteoclast activity resulting from orthopedic or dental implants, and/or age-related osteolysis.

It is now disclosed that the pentapeptide 5aa-OGP is a potent CB2 peptidic agonist that maintains a CB2 tone throughout life and protects against bone loss. Without wishing to be bound by any theory or mechanism of action, it is postulated that 5aa-OGP, acting through CB2 receptor, stimulates osteoblasts (bone formation) and inhibits differentiation and/or activity of osteoclasts (bone resorption), resulting in a net positive effect on bone mass. The 5aa-OGP was found to be efficient in treating osteolysis, such as inflammation-induced osteolysis. The 5aa-OGP may be used as a stand-alone treatment.

Advantageously, the pentapeptide 5aa-OGP was found to act as a positive allosteric modulator, enabling its joint activity with additional CB2 agonists.

The present invention further provides pharmaceutical compositions comprising the active form of OGP and a non-psychoactive cannabinoid, and methods for treating bone fractures. In some specific embodiments of the current invention, the pharmaceutical compositions comprise the pentapeptide H4(99-103) (5aa-OGP) and CBD. The compositions of the present invention may beneficially be used for accelerating healing of bone fractures resulting from injuries or from surgical operations. According to some embodiments the fractures are surgical fractures.

It is now disclosed that the combination of pentapeptide 5aa-OGP and CBD achieves improved efficacy. While CBD improves the quality of the newly formed bone by enhancing collagen crosslinking and stabilization, the 5aa-OGP peptide augments bone quantity (trabecular density) and connectivity. Each compound alone was shown to improve the biomechanical strength of the healing bone via a different structural mechanism. Without wishing to be bound by any theory or mechanism of action, it is postulated that the combination of 5aa-OGP and CBD produces a synergistic effect in accelerating bone growth and repair, and improving newly formed bone quality and strength. According to some embodiments, the combination of pentapeptide 5aa-OGP and CBD acts synergistically, giving improved results over each of the active agents when used alone.

According to one aspect, the present invention provides a peptide comprising an amino acid sequence YGFGG (SEQ ID NO:1; denoted herein 5aa-OGP), or an analog or derivative thereof, for use in treating osteolysis.

According to some embodiments, the peptide is for use in treating or preventing inflammation-induced osteolysis.

According to some embodiments, the peptide is for use in treating or preventing aseptic osteolysis.

According to some embodiments, the peptide is for use in treating chemical-induced osteolysis or compound particle-induced osteolysis.

According to some embodiments, the osteolysis is metal particle-induced osteolysis. According to some embodiments, the peptide is for use in treating titanium particle-induced osteolysis.

According to some embodiments, the peptide is for use in treating or preventing osteolysis in periodontal-related disease or condition. According to certain embodiments the periodontal-related disease is periodontitis or peri-implantitis.

According to some embodiments, the peptide is for use in treating or preventing periodontal bone loss.

According to some embodiments, the peptide inhibits osteoclast activity or differentiation while activating osteoblasts.

According to some embodiments, the peptide inhibits bone loss around orthopedic implants. According to other embodiments, the peptide inhibits bone loss around dental or other oral implants. According to additional embodiments, the peptide inhibits bone loss induced by pathogens, metal particles, metal ions/corrosion, wear debris, or trauma.

According to other embodiments, the peptide is for use in an osteoclasis procedure. According to other embodiments, the peptide is for use in a bone osteotomy procedure.

According to some embodiments, the peptide is an active form of osteogenic growth peptide (OGP).

According to some embodiments, the peptide length is up to 50 amino acids residues. According to certain embodiments, the peptide length is up to 45, 40, 35, 30, 25, or 20 amino acid residues. Each possibility represents a separate embodiment of the invention.

According to some embodiments, the peptide is 5-14 amino acid residues in length. According to some embodiments, the peptide is 5-12 amino acid residues in length. According to some embodiments, the peptide is 5-10 amino acid residues in length. According to additional embodiments, the peptide is of 5, 6, 7, 8, 9, or 10 amino acid residues in length. According to specific embodiment, the peptide is a pentapeptide consisting of the sequence set forth in SEQ ID NO: 1 (H4(99-103) or 5aa-OGP).

According to other embodiments, the peptide is the osteogenic growth peptide (OGP) (ALKRQGRTLYGFGG—SEQ ID NO: 2; also denoted herein OGP(1-14)).

According to some embodiments, the peptide is a synthetic peptide.

According to some embodiments, the peptide analog has one addition, deletion, or substitution. According to specific embodiments, the peptide analog has one addition, deletion, or substitution compared to SEQ ID NO:1. According to certain embodiments, the peptide analog has one addition, deletion, or substitution compared to SEQ ID NO:2. According to additional embodiments, the peptide analog comprises the sequence YGFGG and one addition, deletion, or substitution compared to SEQ ID NO:2.

According to some embodiments, the peptide comprises at least one non-natural amino acid residue. According to certain embodiments, the peptide comprises at least one D-amino acid.

According to some embodiments, the peptide is a cyclic peptide.

According to some embodiments, the amino terminus of the peptide is modified. According to certain embodiments, the N-terminus is acylated. According to other embodiments, the carboxy terminus of the peptide is modified. According to certain embodiments, the C-terminus is amidated or esterified, or reduced to an alcohol. According to some embodiments, both N- and C-termini of the peptides are modified. According to other embodiments, both N- and C-termini of the peptides are non-modified.

According to some embodiments, the peptide comprises a moiety that stabilizes the peptide. According to certain embodiments, the peptide is modified with a moiety that enhances the permeability of the peptide.

According to some embodiments, the N- and/or C-terminus are modified with a moiety that stabilizes the peptide. According to certain embodiments, the N- and/or C-terminus are modified with a moiety that enhance the permeability of the peptide.

A conjugate or multimer comprising the peptide described herein is provided according to certain embodiments of the invention.

According to some embodiments, the peptide is attached to a moiety selected from the group consisting of a fatty acid moiety, a proteinaceous moiety and a combination thereof. According to certain embodiments, the fatty acid moiety comprises a myristoyl fatty acid and the proteinaceous moiety comprises at least one positively charged amino acid.

A pharmaceutical composition comprising the peptide described herein and an acceptable carrier, excipient or diluent is provided according to another aspect.

According to some embodiments, the pharmaceutical composition comprises a peptide as described herein in a dosage ranging from 0.5 and 500 μg. According to additional embodiments, the pharmaceutical composition comprises a peptide as described herein in a dosage ranging from about 5 to about 250 μg.

According to some embodiments, the pharmaceutical composition comprises the peptide or analog described herein in a concentration of 1 to 500 μg/mL According to certain embodiments, the pharmaceutical composition comprises the peptide or analog described herein in a concentration of 2 to 400 μg/mL According to certain embodiments, the pharmaceutical composition comprises the peptide or analog described herein in a concentration of 20 to 300 μg/mL According to certain embodiments, the pharmaceutical composition comprises the peptide or analog described herein in a concentration of 50 to 200 μg/mL.

According to some embodiments, the pharmaceutical composition is formulated for oral, parenteral or topical administration.

According to some embodiments, the pharmaceutical composition is formulated for injectable administration.

According to some embodiments, the pharmaceutical composition is formulated in a dosage form selected from the group consisting of solutions, powders, granules, elixirs, tinctures, suspensions, emulsions and gels. According to some embodiments, the pharmaceutical composition is in a form of cream, foam, gel, lotion, mouthwash, toothpaste, or ointment. Each possibility represents a separate embodiment of the invention. According to additional embodiments, the pharmaceutical composition is formulated as a capsule, a tablet, a liquid, or a syrup. According to certain embodiments, the pharmaceutical composition is in a form of toothpaste.

According to an aspect, the present invention provides a method of treating osteolysis, comprising administering to a subject in need of such treatment a pharmaceutical composition comprising a therapeutically effective amount of a peptide comprising of an amino acid sequence YGFGG or an analog or derivative thereof.

According to some embodiments, the osteolysis is inflammation-related osteolysis.

The pharmaceutical composition, the peptide and the uses are as described hereinabove.

According to some embodiments, the method comprises integrating an implant. According to specific embodiments, the method comprises integrating a titanium implant.

According to certain embodiments, the inflammation-related osteolysis is a periodontal-related disease or disorder.

According to some embodiments, the osteolysis is particle-induced osteolysis. According to certain exemplary embodiments, the osteolysis is titanium particle-induced osteolysis.

According to some embodiments, the method comprises a dental implant integration. According to other embodiments, the method comprises an orthopedic implant integration.

According to some embodiments, the subject is a mammal. According to certain embodiments, the subject is a human subject.

According to some embodiments, the method reduces bone inflammation.

According to some embodiments, the route of administration is selected from the group consisting of orally, topically, locally, subcutaneously, intravenously, intramuscularly, intranasally, sublingual, transdermal, or intradermally. According to some embodiments, the route of administration is parenteral. According to specific embodiments, the pharmaceutical composition is administered directly, or adjacent to the bone. According to other embodiments the route of administration is selected from the group consisting of subcutaneously, intravenously, intramuscularly, intradermally or via inhalation. According to alternative embodiments the administration may be oral. According to additional embodiments the administration is topical.

According to some embodiments, the pharmaceutical composition is administered at least twice a day, once a day, twice a week or once a week. According to some embodiments, the pharmaceutical composition is administered at least once a day. According to other embodiments, the administration is limited to the surgical site during, before or after the surgical procedure.

According to some embodiments, the method comprises administering the pharmaceutical composition at a daily dose comprising from about 0.5 to about 500 μg/day of the peptide described herein. According to other embodiments, the method comprises administering said composition at a daily dose comprising from about 5 to about 250 μg/day of the peptide described herein.

According to some embodiments, the peptide is administered using a delivery vehicle. Delivery vehicles for the peptide in the pharmaceutical compositions include in non-limiting examples, naturally occurring polymers, microparticles, nanoparticles, and other macromolecular complexes capable of mediating delivery of the peptide to a tissue and/or a host cell. Vehicles can also comprise other components or functionalities that further modulate, or that otherwise provide beneficial properties.

According to an additional aspect, the present invention provides a peptide comprising the amino acid sequence YGFGG (5aa-OGP), or an analog or derivative thereof for use as a selective agonist of CB2 receptor or as a positive allosteric modulator of CB2 receptor.

According to some embodiments, the peptide is for use in a combination with an additional CB2 agonist. According to additional embodiments, the combination is for used in treating or preventing osteolysis cases as described hereinabove.

According to another aspect, the present invention provides a pharmaceutical composition comprising: (1) a peptide comprising an amino acid sequence YGFGG (SEQ ID NO:1), or an analog or derivative thereof, and (2) a non-psychoactive cannabinoid, derivative or analog thereof, and a carrier, diluent or excipient.

According to some embodiments, the non-psychoactive cannabinoid is selected from the group consisting of cannabidiol (CBD), cannabigerol (CBG), cannabichromene (CBC) and cannabidivarin (CBDV). According to certain exemplary embodiments, the non-psychoactive cannabinoid is CBD.

According to some embodiments, the non-psychoactive cannabinoid is obtained from a natural source.

The peptide is as described herein above. According to certain embodiments, the peptide is an active form of osteogenic growth peptide (OGP). According to additional embodiments, the peptide is of 5, 6, 7, 8, 9, or 10 amino acid residues in length. According to specific embodiment, the peptide is a pentapeptide consisting of the sequence set forth in SEQ ID NO: 1 (H4(99-103) or 5aa-OGP). According to other embodiments, the peptide is the osteogenic growth peptide (OGP) (ALKRQGRTLYGFGG—SEQ ID NO: 2; also denoted herein OGP(1-14)). According to some embodiments, the peptide is a synthetic peptide.

According to some embodiments, the peptide and the non-psychoactive cannabinoid are present in a single pharmaceutical composition. According to other embodiments, the peptide and the non-psychoactive cannabinoid, derivative or analog thereof are present in separate pharmaceutical compositions.

According to some embodiments, the pharmaceutical composition further comprises a non-psychoactive cannabinoid or a cannabis extract. According to specific embodiments the non-psychoactive cannabinoid is cannibidiol (CBD). According to certain embodiments, the pharmaceutical composition further comprises a cannabinoid selected from the group consisting of cannabinol (CBN), cannabigerol (CBG), cannabichromene (CBC), cannabicyclol (CBL), cannabivarin (CBV), tetrahydrocannabivarin (THCV), cannabidivarin (CBDV), cannabichromevarin (CBCV), cannabigerovarin (CBGV), tetrahydrocannabinol (THC), tetrahydrocannabinolic acid (THCA), cannabigerol monomethyl ether (CBGM), derivatives thereof, analogs thereof and any combination thereof. Each possibility represents a separate embodiment of the invention. According to certain specific embodiments, the CBD derivative is CBDA.

According to some embodiments, the pharmaceutical composition does not contain THC.

According to other embodiments, the pharmaceutical composition comprises THC at a concentration lower than 0.2%.

According to some embodiments, the pharmaceutical composition comprises CBD in a dosage ranging from 0.5 to 500 mg. According to some embodiments, the pharmaceutical composition comprises a peptide as described herein in a dosage ranging from 0.5 and 500 μg. According to other embodiments, the pharmaceutical composition comprises CBD in a dosage ranging from about 1 to about 200 mg. According to additional embodiments, the pharmaceutical composition comprises a peptide as described herein in a dosage ranging from about 5 to about 250 μg.

According to specific embodiments, the pharmaceutical composition comprises CBD in a dosage ranging from between 0.5 and 500 mg and a peptide as described herein in a dosage ranging from between 0.5 and 500 μg.

According to some embodiments, the pharmaceutical composition is formulated for injectable administration.

According to some embodiments, the pharmaceutical composition is formulated in a dosage form selected from the group consisting of solutions, powders, granules, elixirs, tinctures, suspensions, emulsions and gels.

According to some embodiments, the pharmaceutical composition is for use in acceleration of bone formation and regeneration, bone growth and/or bone fracture repair. According to some embodiments, the pharmaceutical composition is for use in treating a bone fracture. According to some embodiments, the pharmaceutical composition is for use in accelerating bone healing. According to other embodiments, the pharmaceutical composition is for use in an osteoclasis procedure. According to other embodiments, the pharmaceutical composition is for use in a bone osteotomy procedure. According to some embodiments, the pharmaceutical composition is for use in treating osteolysis.

According to an aspect, the present invention provides a method of accelerating of bone formation and regeneration, bone growth and/or bone fracture repair, comprising administering to a subject in need of such treatment a therapeutically effective amount of a peptide comprising of an amino acid sequence YGFGG or an analog or derivative thereof, and a non-psychoactive cannabinoid, derivative or analog thereof.

According to another aspect, the present invention provides a method of treating a bone fracture or condition comprising administering to a subject in need of such treatment a therapeutically effective amount of a peptide comprising of an amino acid sequence YGFGG (SEQ ID NO: 1) or an analog thereof, and a non-psychoactive cannabinoid, derivative or analog thereof.

According to some embodiments, the non-psychoactive cannabinoid is CBD, a CBD derivative or an analog thereof.

According to some embodiments, the peptide and the non-psychoactive cannabinoid are co-formulated. According to some embodiments, the peptide and the non-psychoactive cannabinoid are present within a single pharmaceutical composition. According to some embodiments, the peptide and the non-psychoactive cannabinoid are co-formulated in a pharmaceutical composition further comprising at least one carrier, diluent or excipient.

According to some embodiments, the method comprising administering a pharmaceutical composition comprising a peptide described herein, and a pharmaceutical composition comprising the non-psychoactive cannabinoid.

According to some embodiments, the administering of the peptide and the non-psychotropic cannabinoid is carried out substantially simultaneously, concurrently, alternately, sequentially or successively. Each possibility represents a separate embodiment of the invention.

According to some embodiments, the method comprising administering a pharmaceutical composition comprising the peptide and the non-psychoactive cannabinoid, derivative or analog thereof, and a pharmaceutically acceptable carrier, excipient or diluent.

The peptide and the non-psychoactive cannabinoid or derivative thereof are as described hereinabove. According to certain embodiments, the non-psychoactive cannabinoid is CBD.

According to some embodiments, the subject is a mammal. According to certain embodiments, the subject is a human subject.

According to some embodiments, the treatment increases bone formation.

According to some embodiments, the treatment reduces malunion or nonunion of the fractured bone.

According to some embodiments, the method accelerates bone regeneration. According to some embodiments, the method reduces bone inflammation.

According to some embodiments, the route of administration is parenteral. According to specific embodiments, the pharmaceutical composition is administered directly, or adjacent to the fracture site. According to other embodiments the route of administration is selected from the group consisting of subcutaneously, intravenously, intramuscularly, intradermally or via inhalation. According to alternative embodiments the administration may be oral or intranasal.

According to certain embodiments, the peptide and the non-psychoactive cannabinoid are administered in different routes of administration.

According to some embodiments, the pharmaceutical composition is administered at least twice a day, once a day, twice a week or once a week. According to some embodiments, the pharmaceutical composition is administered at least once a day. According to other embodiments, the administration is limited to the surgical site during, before or after the surgical procedure.

According to some embodiments, the method provides from about 0.2 to about 2 mm/day bone growth rate. According to other embodiments, the method provides from about 0.5 to about 1.5 mm/day bone growth rate. According to further embodiments, the method provides enhancement of a functional outcome following the bone fracture.

According to some embodiments, the pharmaceutical composition is administered before a surgery that includes displacement or damage to the bone. According to certain embodiments, the pharmaceutical composition is administered before a surgery to repair and/or heal a broken bone. According to certain embodiments, the pharmaceutical composition is administered at least one day before a surgery that includes displacement or damage to the bone. According to additional embodiments, the pharmaceutical composition is administered between 2 and 5 days before a surgery that includes displacement or damage to the bone. According to certain embodiments, the pharmaceutical composition is administered during the surgery. According to other embodiments, the pharmaceutical composition is administered after the surgery.

According to certain embodiments, the bone fracture is a result of a distraction osteogenesis procedure. According to some embodiments, the composition is administered during the latency phase of the distraction osteogenesis procedure. According to other embodiments, the composition is administered during the distraction phase of the distraction osteogenesis procedure. According to additional embodiments, the composition is administered during the consolidation phase of the distraction osteogenesis procedure. According to further embodiments, the composition is administered during the latency, distraction and consolidation phases of the distraction osteogenesis procedure.

According to some embodiments, the pharmaceutical composition is administered starting at least two days before the bone dissection of the distraction osteogenesis procedure. According to other embodiments, the pharmaceutical composition is administered starting at least five days before the bone dissection of the distraction osteogenesis procedure.

According to certain embodiments, the method comprises accelerating bone consolidation following bone distraction. According to other embodiments, the method comprises accelerating bone consolidation following bone dissection.

According to some embodiments, the bone condition comprises an osseointegration procedure. According to further embodiments, the method comprises administering the composition following the implant insertion stage of the osseointegration procedure, following the abutment insertion stage of the osseointegration procedure or a combination thereof. Each possibility represents a separate embodiment of the invention. According to some embodiments, the osseointegration procedure comprises a dental implant integration. According to further embodiments, the invention provides a method of accelerating bone growth in the osseointegration procedure of a dental implant in jawbone. According to other embodiments, the osseointegration procedure comprises an orthopedic implant integration. According to some embodiments, the method comprises reducing the time of the implant's osseointegration.

According to some embodiments, the invention provides a method of accelerating bone growth in a subject having a pathological fracture. According to further embodiments, the pathological fracture comprises a fracture associated with osteoporosis, osteomalacia, malignant tumor, multiple myeloma, osteogenesis imperfecta congenita, cystic bone, suppurative myelitis, osteopetrosis, or a combination thereof. Each possibility represents a separate embodiment of the invention.

According to some embodiments, the invention provides a method of accelerating bone growth in a subject having a fracture by external force. According to other embodiments, the invention provides a method of accelerating bone repair in a subject having a fracture by external force.

According to further embodiments, the bone fractures comprise fractures of humerus, ulna, radius, femur, tibia, fibula, patella, ankle bones, wrist bones, carpals, metacarpals, phalanges, tarsals, metatarsals, ribs, sternum, vertebrae, scapula, clavicle, pelvis, sacrum or craniofacial bones. Each possibility represents a separate embodiment of the invention. According to additional embodiments, the composition is administered to a subject having a non-union fracture, mal-union fracture or delayed union fracture. Each possibility represents a separate embodiment of the invention.

According to some embodiments, the method comprises administering the pharmaceutical composition at a daily dose comprising from about 0.5 to about 500 mg/day CBD and from about 0.5 to about 500 μg/day of the peptide described herein. According to other embodiments, the method comprises administering said composition at a daily dose comprising from about 1 to about 200 mg/day CBD and about 5 to about 250 μg/day of the peptide described herein.

It is to be understood that any combination of each of the aspects and the embodiments disclosed herein is explicitly encompassed within the disclosure of the present invention.

Further embodiments and the full scope of applicability of the present invention will become apparent from the drawings and detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### DRAWINGS

FIGS. 1A-1E depict interaction of the 5aa-OGP peptide with the allosteric site of human CB2 receptor. FIGS. 1A and 1B represent conformation of the peptide when 5aa-OGP is bound to the extracellular surface in the absence (FIG. 1A) and presence (FIG. 1B) of CP55,940. The conformations presented are taken from the last step of a Molecular dynamics simulation (MD). Residues forming direct hydrogen bonds with the peptide and the respective interactions are highlighted in purple. Residues forming hydrophobic interactions are labelled in grey color. The Amino-π interaction is labelled in orange color. FIG. 1C—Competitive binding assay between 5aa-OGP and tritiated CP55940. CHO-CB2-derived membranes were used for a CB2 binding assay. No detection of CP55,940 chase-out in the presence of increasing concentrations of 5aa-OGP indicates that 5aa-OGP and CP55940 do not compete over the same binding site at CB2. (FIGS. 1D, 1E) Effect of 5aa-OGP on the stimulation of binding of ^{[35S]}GTPγS induced by CP55940. CHO-CB2-derived membranes were used in a ^{[35S]}GTPγS binding assay. The white bar shows the relative activation of CB2 by 0.1 nM (FIG. 1D) or 10 nM (FIG. 1E) of CP55,940 above basal levels (DMSO only) in the absence of 5aa-OGP. Other graphs show relative activation of CB2 induced by CP55,940 in the presence of increasing concentrations of OGP. * p<0.05, vs. CP55940 only (no 5aa-OGP).

FIGS. 2A-2F show that the effect of 5aa-OGP in bone cells is dependent on CB2. FIGS. 2A-2B—Proliferative activity of HU910, a synthetic CB2 selective agonist (FIG. 2A), and of 5aa-OGP (FIG. 2B) in WT and CB2^{-/-}-derived murine osteoblasts. Data are the mean±SD obtained in triplicate and were repeated at least 2 times. *p<0.05 vs. Veh in the same genotype, non-parametric 1-way ANOVA. FIGS. 2C-2D—The proliferative activity of HU910 (FIG. 2C) and 5aa-OGP (FIG. 2D) in human osteoblasts is abrogated by the CB2-selective antagonist SR144528 (SR2). Data are mean±SD obtained in triplicate. *p<0.05 vs. Veh, #p<0.05 vs. the CB2 agonist with no SR2, non-parametric 1-way ANOVA. FIGS. 2E-2F—5aa-OGP attenuates osteoclastogenesis (number of multinucleated TRAP-positive cells per well) in WT—but not in CB2^{-/-}—derived murine cultures. Data are the mean±SD obtained in six wells per condition. *p<0.05 vs. Veh in the same genotype, non-parametric 1-way ANOVA.

FIGS. 3A-3D. show the effect of 5aa-OGP on bone recovery in an OVX model. Eight-week old mice were OVX (or Sham), and treatment with 5aa-OGP or vehicle started after 6 weeks for 6 weeks. FIG. 3A—trabecular bone volume fraction (BV/TV); FIG. 3B—trabecular volumetric bone mineral density (vBMD); FIG. 3C—Trabecular number (Tb.N); FIG. 3D—Trabecular thickness (Tb.Th). #, p<0.05 vs Sham; *, p<0.05 vs OVX; +, p=0.051 vs OVX.

FIGS. 4A-4C. show OGP(1-14) levels in women and the effect of 5aa-OGP on age-related bone loss in male mice. FIG. 4A—Immuno-reactive (ir) OGP(1-14) levels were measured in serum from human female subjects aged 18 to 49 years. irOGP(1-14) levels were significantly lower in the 5th decade compared with the 3rd and 4th decades of life. Data are the mean±SD. n=28 (3rd decade), n=6 (4th decade), n=6 (5th decade). *p<0.05 vs. 5th decade aged women, Kruskal-Willis one-way ANOVA and Mann Whitney-Wilcoxon rank sum tests. FIG. 4B—Exogenous administration of 5aa-OGP prevents age-related decline in trabecular bone volume fraction (BV/TV) in the distal femur of 6-month old male mice. Data are the mean±SD, n=8. *p<0.05 vs. vehicle (Veh)-treated mice, 1-way ANOVA. FIG. 4C—Representative pCT images of the distal femur from each group. Bar=0.5 mm.

FIGS. 5A-5C show the effect of topically administered 5aa-OGP on Ti particle-induced osteolysis in vivo: Fibrin membranes including titanium particles derived from sand-blasted Ti implants, with 5aa-OGP (TiP+5aa-OGP) or with saline (TiP) were implanted onto mice calvaria. Membranes with no TiP were inserted in the control group. Mice were sacrificed 4 weeks post-op. and subjected to CT analysis. FIG. 5A—Pit Resorption Volume over total volume (PR.V/TV), FIG. 5B—Bone volume in the region of interest, chiefly representing the calvarial thickness), and FIG. 5C—Representative CT images of the calvaria. Data are expressed as mean±SD (n=4). *p<0.05 vs. Titanium.

### DETAILED DESCRIPTION OF THE INVENTION

The present invention provides pharmaceutical compositions and methods for use in treating osteolysis, in particular inflammation-induced osteolysis. The pharmaceutical compositions of the invention comprise a peptide comprising the sequence YGFGG or analogs thereof.

The present invention further provides pharmaceutical compositions and methods for accelerating bone growth or repair, increase bone regeneration, and/or healing bone fractures. The pharmaceutical compositions of the invention comprise a peptide comprising the sequence YGFGG and non-psychoactive cannabinoid. The combination of the peptide and the non-psychoactive cannabinoid in some embodiments provides a synergistic effect. According to certain exemplary embodiments, the non-psychoactive cannabinoid is CBD.

As used herein, “synergy” or “synergistic” interchangeably refer to the combined effects of two active agents that are greater than their additive effects. Synergy can also be achieved by producing an efficacious effect with combined inefficacious doses of two active agents.

The pentapeptide H4(99-103) (denoted herein 5aa-OGP) is an active peptide having the sequence YGFGG (SEQ ID NO:1). The peptide is a proteolytically cleaved fragment of the Osteogenic Growth Peptide (OGP) peptide (SEQ ID NO: 2; also denoted OGP(1-14)).

The term “analog”, as used herein, refers to a sequence that possesses a similar or identical function as a parent peptide but has modifications or non-natural amino acids in its sequence. The term analog further refers to a peptide that possesses a similar or identical function as the parent peptide but need not necessarily comprise an amino acid sequence that is identical to the amino acid sequence of the parent peptide, or possesses a structure that is similar or identical to that of the parent peptide.

The term “pharmaceutical composition” as used herein refers to any composition comprising at least one pharmaceutically active ingredient, formulated such that it facilitates accessibility of the active ingredient to the target organ. The term “pharmaceutical composition” further includes a product that results from the mixing or combining of more than one active ingredient and includes both fixed and non-fixed combinations of the active ingredients. The term “fixed combination” means that the active ingredients, e.g. a compound disclosed herein, or a pharmaceutically acceptable salt thereof, and a co-agent, are both administered to a patient simultaneously in the form of a single entity or dosage. The term “non-fixed combination” means that the active ingredients, e.g. a compound disclosed herein, or a pharmaceutically acceptable salt thereof, and a co-agent, are administered to a patient as separate entities either simultaneously, concurrently or sequentially with no specific intervening time limits, wherein such administration provides effective levels of the two compounds in the body of the patient. The latter also applies to cocktail therapy, e.g. the administration of three or more active ingredients.

The term “pharmaceutically acceptable” means approved by a regulatory agency of the Federal or a state government or listed in the U. S. Pharmacopeia or other generally recognized pharmacopeia for use in animals, and more particularly in humans.

The term “carrier” as used herein indicates an inactive substance that serves as mechanisms to improve the delivery and the effectiveness of drugs and can be identified by a skilled person in view of the route of administration and related composition formulation.

The term “excipient” as used herein indicates an inactive substance that can be used any of various media acting usually as coloring agents, preservatives, coatings, solvents, binders or diluents to bulk up formulations that contain active ingredients (thus often referred to as “bulking agents,” “fillers,” or “diluents”), to allow convenient and accurate dispensation of a drug substance when producing a dosage form. Suitable excipients can include any substance that can be used to bulk up formulations with the peptide described herein to allow for convenient and accurate dosage.

The singular forms “a”, “an”, and “the” include plural referents unless the context clearly dictates otherwise.

According to an aspect, the present invention provides a peptide comprising an amino acid sequence YGFGG (SEQ ID NO:1; denoted herein 5aa-OGP), or an analog or derivative thereof, for use in treating osteolysis

According to an aspect, the present invention provides a pharmaceutical composition comprising: (1) a peptide comprising of an amino acid sequence YGFGG (SEQ ID NO:1) or an analog thereof, and (2) CBD, a CBD derivative or analog thereof, and a carrier, diluent or excipient.

According to an aspect, the present invention provides a pharmaceutical composition comprising: (1) a pentapeptide consisting of an amino acid sequence YGFGG (SEQ ID NO:1) or an analog thereof, and (2) CBD, a CBD derivative or analog thereof, and a carrier, diluent or excipient.

According to an additional aspect, the present invention provides a pharmaceutical composition comprising: (1) a pentapeptide consisting of an amino acid sequence YGFGG (SEQ ID NO:1), and (2) CBD, and a carrier, diluent or excipient.

According to an additional aspect, the present invention provides a pharmaceutical combination comprising: (1) a peptide comprising of an amino acid sequence YGFGG (SEQ ID NO:1) or an analog thereof, and (2) non-psychoactive cannabinoid, a derivative or analog thereof.

The peptide and non-psychoactive cannabinoid are as described herein. According to certain embodiments, the non-psychoactive cannabinoid is CBD.

The term “pharmaceutical combination” as used herein refers to either a pharmaceutical composition comprising one or more active pharmaceutical ingredient and one or more second therapeutic compounds or a pharmaceutical composition comprising an active pharmaceutical ingredient co-administered with a second therapeutic compound.

According to some embodiments, the peptide and the non-psychoactive cannabinoid are present in the same pharmaceutical composition. According to other embodiments, the peptide and the non-psychoactive cannabinoid described herein are present in separate pharmaceutical compositions.

According to some embodiments, the terms “pharmaceutical composition” and “pharmaceutical combination” are used herein interchangeably.

According to some embodiments, the peptide length is up to 50 amino acids residues. According to certain embodiments, the peptide length is up to 45, 40, 35, 30, 25, or 20 amino acid residues. Each possibility represents a separate embodiment of the invention.

According to some embodiments, the peptide is 5-14 amino acid residues in length. According to some embodiments, the peptide is 5-13 amino acid residues in length. According to some embodiments, the peptide is 5-12 amino acid residues in length. According to some embodiments, the peptide is 5-11 amino acid residues in length. According to some embodiments, the peptide is 5-10 amino acid residues in length. According to additional embodiments, the peptide is of 5, 6, 7, 8, 9, 10, 11, 12, 13, 14, 15, 16, 17, 18, 19, or 20 amino acid residues in length. Each possibility represents a separate embodiment of the invention. According to specific embodiment, the peptide is a pentapeptide consisting of the sequence set forth in SEQ ID NO: 1 (also denoted herein H4(99-103) or 5aa-OGP).

According to some embodiments, the peptide described herein is modified. Modifications can be made directly to the peptide, such as by glycosylation, side chain oxidation, phosphorylation, addition of a linker molecule, addition of a fatty acid, nucleic acid, amino acid substitution and the like.

Amino acid substitution to produce peptide functional variants preferably involves conservative amino acid substitution i.e., replacing one amino acid residue with another that is biologically and/or chemically similar, e.g., a hydrophobic residue for another, or a polar residue for another.

Modifications also encompass introduction of one or more non-natural amino acids, introduced so as to render a peptide non-hydrolysable or less susceptible to hydrolysis, as compared to the original peptide. The peptides may contain one or more D-amino acids or one or more non-hydrolysable peptide bonds linking amino acids. Alternatively, one may modify the peptides in order to reduce the potential for hydrolysis by proteases. For example, to determine the susceptibility to proteolytic cleavage, peptides may be labeled and incubated with cell extracts or purified proteases and then isolated to determine which peptide bonds are susceptible to proteolysis. Alternatively, potentially susceptible peptide bonds can be identified by comparing the amino acid sequence of a peptide with the known cleavage site specificity of a panel of proteases. Based on the results of such assays, individual peptide bonds which are susceptible to proteolysis can be replaced with non-hydrolysable peptide bonds.

Non-hydrolysable peptide bonds are known in the art, along with procedures for synthesis of peptides containing such bonds. Non-hydrolysable bonds include, but are not limited to, psi[CH₂NH]— reduced amide peptide bonds, psi[COCH₂]— ketomethylene peptide bonds, psi[CH(CN)NH]— (cyanomethylene) amino peptide bonds, psi [CH₂CH(OH)]— hydroxyethylene peptide bonds, psi[CH₂O]— peptide bonds, and psi[CH₂S]— thiomethylene peptide bonds.

According to some embodiments, the peptide has a permeability-enhancing moiety. The permeability-enhancing moiety may be connected to any position in the peptide moiety, directly or through a spacer or linker.

Pharmaceutically acceptable excipients used in the compositions of the present invention are selected from but not limited to the group of excipients generally known to persons skilled in the art e.g. vehicles, bulking agents, stabilizers, preservatives, surfactants, hydrophilic polymers, solubility enhancing agents such as glycerin, various grades of polyethylene oxides, beta-cyclodextrins like sulfobutylether-beta-cyclodextrin, transcutol and glycofurol, tonicity adjusting agents, local anesthetics, pH adjusting agents, antioxidants, osmotic agents, chelating agents, viscosifying agents, wetting agents, emulsifying agents, acids, sugar alcohol, reducing sugars, non-reducing sugars and the like, or mixtures thereof.

The pharmaceutical compositions can further comprise pharmaceutical excipients including, but not limited to, sodium chloride, potassium chloride, magnesium chloride, sodium gluconate, sodium acetate, calcium chloride, sodium lactate, and the like. The composition, if desired, can also contain minor amounts of sugar alcohols, wetting or emulsifying agents, and pH adjusting agents. Antibacterial agents such as benzyl alcohol or methyl parabens; antioxidants such as ascorbic acid or sodium bisulfite; chelating agents such as ethylenediaminetetraacetic acid; and agents for the adjustment of tonicity such as sodium chloride or dextrose are also envisioned.

Pharmaceutical compositions for parenteral administration can also be formulated as suspensions of the active compounds. Such suspensions may be prepared as oily injection suspensions or aqueous injection suspensions. For oily suspension injections, suitable lipophilic solvents or vehicles can be used including fatty oils such as sesame oil, or synthetic fatty acids esters such as ethyl oleate, triglycerides or liposomes. Aqueous injection suspensions may contain substances which increase the viscosity of the suspension, such as sodium carboxymethyl cellulose, sorbitol or dextran. Optionally, the suspension may also contain suitable stabilizers or agents which increase the solubility of the compounds, to allow for the preparation of highly concentrated solutions.

According to some embodiments, the peptide analog is as described in U.S. Pat. No. 6,479,460. According to certain embodiments, the peptide is an analog having the general formula:

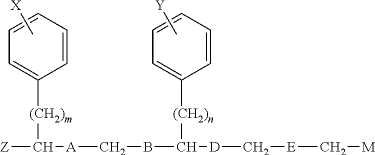

wherein
A, B, D and E, which may be the same or different, represent CONH, CH₂NH, CH₂S, CH₂O, NHCO, N(CH₃)CO, (CH₂)₂, CH═CH, C(O)CH₂, CH₂SO or C(O)O, M represents C(O)OH, CH₂OH, C(O)NH₂, C(O)OCH₃, CH₂OCH₃, H, C(O)NHCH₃, or C(O)N(CH₃)₂,
Z represents NH₂, H, NHCH₃, N(CH₃)₂, OH, SH, OCH₃, SCH₃. C(O)OH, C(O)NH₂, C(O)OCH₃, C(O)NHCH₃ or C(O)N(CH₃)₂,
n and m each represent an integer of 1 to 6,
X and Y, if in the ortho or para positions, each represent OH, OCH₃, F, Cl, Br, CF₃, CN, NO₂, NH₂, NHCH₃, N(CH₃)₂, SH, SCH₃, CH₂OH, NHC(O)CH₃, C(O)OH, C(O)OCH₃, C(O)NH₂, C(O)NHCH₃, C(O)N(CH₃)₂, or CH₃, and
Y, if in the para or meta positions, represents C(O)C₆H₅, C(O)CH₃, C₆H₅,
CH₂C₆H₅, and, if in the ortho or para positions can additionally represent C(O)C₆H₅, C(O)CH₃, C₆H₅, CH₂C₆H₅, CH₂CH₃, CH(CH₃)₂, or C₆H₁₁.

According to certain embodiments, the analog is desaminoTyr-Gly-Phe-Gly-Gly.

According to certain embodiments, the analog is desaminoTyr-Gly-N(CH₃)—CH(CH₂C₆H₅)—C(O)-Gly-Gly. According to certain embodiments, the analog is desaminoCH(CH₂C₆H₅OH)—CH₂-Gly-Phe-Gly-Gly. According to certain embodiments, the analog is desaminoTyr-NH—CH₂—CH₂-Phe-Gly-Gly. According to certain embodiments, the analog is desaminoTyr-Gly-NH—CH(CH₂C₆H₅)—CH₂-Gly-Gly. According to certain embodiments, the analog is desaminoTyr-Gly-Phe-NH—CH₂—CH₂-Gly. According to certain embodiments, the analog is desaminoTyr-Gly-Phe-NH—CH₂—CH₂—CH₂—CH₂—C(O)—OH. According to certain embodiments, the analog is Tyr-Gly-NH—CH(CH₂C₆H₄—(C(O)—C₆H₅))—C(O)-Gly-Gly. According to certain embodiments, the analog is Tyr(m-I)-Gly-NH—CH(CH₂C₆H₄(C(O)C₆H₅))C(O)-Gly-Gly.

### Methods of Treatment

According to an aspect, the present invention provides a method of treating osteolysis comprising administering to a subject in need of such treatment a therapeutically effective amount of a pharmaceutical composition comprising a peptide comprising an amino sequence set forth in SEQ ID NO: 1, or an analog thereof, and a pharmaceutically acceptable carrier, excipient or diluent.

According to another aspect, the present invention provides a method of treating a bone fracture or condition comprising administering to a subject in need of such treatment a therapeutically effective amount of a pharmaceutical composition comprising a peptide comprising an amino sequence set forth in SEQ ID NO: 1, or an analog thereof, and CBD, a derivative or analog thereof, and a pharmaceutically acceptable carrier, excipient or diluent.

The peptide and CBD are as described herein.

The term “therapeutically effective amount” of the compound is that amount of a composition containing the peptide according to the present invention which is sufficient to provide a beneficial effect to the subject to which the compound is administered. An effective amount of the compound may vary according to factors such as the disease state, age, sex, and weight of the individual.

The term “effective amount” as used herein refers to a sufficient amount of the compositions comprising the peptide described herein to prevent or stop bone loss at a reasonable benefit/risk ratio applicable to any medical treatment.

According to additional embodiments, the invention provides a method of treating or preventing bone loss following an implant procedure, the method comprising administering to said subject an effective amount of the pharmaceutical composition of the invention.

According to some embodiments, the implant is a dental implant. According to additional embodiments, the implant is an orthopedic implant.

According to additional embodiments, the invention provides a method of treating or preventing age-related bone loss, the method comprising administering to said subject an effective amount of a composition comprising the peptide described herein.

The term “repair” as used herein refers to the bone fracture healing, including osteogenesis phase.

The term “bone condition” as used herein refers to any disease, condition, disorder, syndrome, or trauma of or to a bone which would be benefited, treated, rescued or healed by osteogenesis. Examples of such bone conditions include, but are not limited to, fracture, fracture by an external force, pathologic fractures, fatigue fractures, delayed unions, non-unions, malunions, distraction osteogenesis, osteotomy and osseointegration.

The term “fracture” as used herein refers to a fracture of a skeletal bone, whether simple or compound.

The term “distraction osteogenesis” as used herein refers to the process of lengthening bones or repairing skeletal deformities comprising increasing the size of a gap between sections of bone and allowing new bone to grow in said gap.

The term “osteotomy” as used herein refers to a surgical sectioning or surgical drilling of bone.

The term “osseointegration” as used herein refers to the firm anchoring of a surgical implant or prosthesis (as in dentistry or in bone surgery) by the growth of bone around or into said surgical implant or prosthesis without fibrous tissue formation at the interface of said bone and said surgical implant or prosthesis.

The terms “osteogenesis” or “bone growth” that can be used interchangeably, as used in some embodiments, refer to an increase in the presence and activity of osteoblasts and the direct formation of bone tissue by said osteoblasts.

According to some embodiments, the bone repair is subsequent to a fracture, selected from fracture by external force, surgical fracture, pathological fracture, and fatigue fracture. According to specific embodiments, the subject in need suffers from a bone fracture, caused by surgical fracture. According to some embodiments, the surgical fracture comprises fractures originating from osteotomy, osteoclasis, and distraction osteogenesis surgeries. According to further embodiments, the bone repair in a subject suffering from bone fracture further comprises enhancing functional outcome following fracture.

According to additional embodiments, the invention provides a method of accelerating bone growth in a subject having a bone fracture selected from the group consisting of a fracture by external force, surgical fracture, pathological fracture, fatigue fracture and a combination thereof, the method comprising orally administering to said subject an effective amount of a composition comprising the pharmaceutical composition of the invention.

The use of the compositions comprising the peptide described herein and CBD is particularly beneficial for the acceleration of bone growth following surgical bone fractures. The compositions of the present invention may be used for treating surgical fractures selected from, but not limited to the fractures originating from osteotomy, osteoclasis, and distraction osteogenesis procedures.

According to some embodiments, the compositions of the present invention are used to accelerate bone growth of a fracture following osteotomy or osteoclasis surgery. Osteotomy and osteoclasis are surgical operations wherein a bone is cut to shorten, lengthen, or change its alignment. Said operations may be performed to correct a deformity of a bone. The non-limiting examples of the osteotomy surgeries are corrections of coxa vara, genu valgum, and genu varum, and osteotomies of the hip, knee, jaw, and chin. Osteotomy may also be performed to straighten a bone that has healed crookedly following a fracture.

According to other embodiments, the compositions of the present invention are used to accelerate bone growth of a fracture following distraction osteogenesis surgery. The distraction osteogenesis procedure is a surgical process used to reconstruct skeletal deformities and lengthen the long bones of the body. A corticotomy is used to fracture the bone into two segments, and the two bone ends of the bone are gradually moved apart during the distraction phase, allowing new bone to form in the gap. When the desired or possible length is reached, a consolidation phase follows in which the bone is allowed to keep healing. Distraction osteogenesis has the benefit of simultaneously increasing bone length and the volume of surrounding soft tissues. The distraction osteogenesis procedure can be used in the field of orthopedics and/or dentofacial orthopedics, for example in lower and upper limb lengthening or in the correction of micrognathia, midface, and fronto-orbital hypoplasia in patients with craniofacial deformities.

According to some embodiments, the composition of the invention is administered to a subject during the latency phase, the distraction phase, the consolidation phase or any combination thereof. Each possibility represents a separate embodiment of the invention.

The administration of the compositions may be started before the distraction osteogenesis surgery. Alternatively, the administration of the compositions may be started on the same day of the distraction osteogenesis surgery. In other embodiments, the administration is started at least one day following the surgery. In alternative embodiments, the compositions are administered starting two, five, ten, fifteen or twenty days from the distraction osteogenesis surgery. Each possibility represents a separate embodiment of the invention.

The compositions of the present invention can be used for accelerating bone consolidation following bone distraction. In these embodiments, the compositions can be administered before the beginning of the consolidation phase or before the beginning of the distraction phase. The compositions of the present invention are further adapted to reduce the consolidation phase following the bone distraction.

The compositions of the present invention can further be used for accelerating bone growth during an osseointegration procedure.

According to some embodiments, the compositions are used for bone growth acceleration in dental implants osseointegration. Additionally or alternatively, the compositions may be used for bone growth acceleration in orthopedic implants osseointegration. The implants may include a variety of biocompatible structures designed to engage the skeletal structure of the body to replace or support a bone structure, including specifically dental implants, craniofacial structures and bone and joint replacement component parts.

The compositions of the present invention may further be used in accelerating bone growth in fractures selected from, but not limited, to the fractures of humerus, ulna, radius, femur, tibia, fibula, patella, ankle bones, wrist bones, carpals, metacarpals, phalanges, tarsals, metatarsals, ribs, sternum, vertebrae, scapula, clavicle, pelvis, sacrum and craniofacial bones. In specific embodiments, distal radius fractures are treated with the compositions of the present invention. Compositions of the present invention may be administered to a subject suffering from any of the following fractures, including fissure fracture, transverse fracture, oblique fracture, spiral fracture, segmental fracture, comminuted fracture, compression fracture, and the like.

The method and the composition of the present invention further provide enhancement of a functional outcome following the bone fracture healing, for example, following the bone fracture of a limb, significant deficits in strength and motion of the limb may appear despite apparently successful repair of the fracture.

According to some embodiments, the pharmaceutical composition is administered orally.

The administration of the compositions may be started before the orthopedic or dental implant surgery. Alternatively, the administration of the compositions may be started on the same day of the orthopedic or dental implant surgery. In other embodiments, the administration is started at least one day following the orthopedic or dental implant surgery. In alternative embodiments, the compositions are administered starting two, five, ten, fifteen or twenty days from the orthopedic or dental implant surgery. Each possibility represents a separate embodiment of the invention.

The compositions disclosed herein may be administered systemically or locally. According to some embodiments, the pharmaceutical composition is formed into a dosage form suitable for intravenous, oral, intranasal, intraarterial, intraperitoneal, transmucosal, topical, or subcutaneous administration.

According to certain embodiments the pharmaceutical composition is formulated for an injectable use. According to other embodiments the pharmaceutical composition is formulated for an oral or inhalation administration use.

According to some embodiments, the compositions are administered at least one time a day. In other embodiments, the compositions are administered 1-4 times a day.

The compositions of the present invention may be administered in combination with other medications for treatment of osteolysis. According to some embodiments, said compositions are administered in combination with minerals. The minerals are selected from Zinc, Copper, Calcium, Phosphorus, Boron and Silicon. The compositions described herein may further be administered in combination with vitamins. The vitamins are selected from, but not limited to, Vitamin C, Vitamin D, or Vitamin E. Vitamin D may comprise Vitamin D₁, Vitamin D₂, Vitamin D₃, Vitamin D₄, Vitamin D₅ or a combination thereof. Separate dosage forms may be administered simultaneously or sequentially or on entirely independent separate regimens.

According to some embodiments, the pharmaceutical composition further comprises an anti-inflammatory agent. According to some embodiments, the anti-inflammatory agent is a non-steroidal anti-inflammatory drug (NSAID). In one embodiment, the NSAID is selected from the group consisting of ibuprofen, flurbiprofen, diclofenac, and naproxen, or a pharmaceutically acceptable salt thereof.

According to some embodiments, the pharmaceutical composition disclosed herein further comprises a tissue adhesive. According to certain embodiments, the tissue adhesive is selected from the group consisting of fibrin(ogen), gelatin, collagen, chitosan, cyanoacrylate, and combinations thereof. Each possibility represents a separate embodiment of the invention.

According to some embodiments, the composition disclosed herein is in a liquid or semi-solid form. Each possibility represents a separate embodiment. In particular embodiments, the composition is in a form of a hydrogel. In other embodiments, the composition disclosed herein is in a form selected from the group consisting of a cream, a paste, a solution, a putty, an emulsion, a suspension, and a powder. Each possibility represents a separate embodiment.

Pharmaceutical compositions for parenteral administration can also be formulated as suspensions of the active compounds. Such suspensions may be prepared as oily injection suspensions or aqueous injection suspensions. For oily suspension injections, suitable lipophilic solvents or vehicles can be used including fatty oils such as sesame oil, or synthetic fatty acids esters such as ethyl oleate, triglycerides or liposomes. Optionally, the suspension may also contain suitable stabilizers or agents which increase the solubility of the compounds, to allow for the preparation of highly concentrated solutions.

According to some embodiments, the pharmaceutical composition comprises a phosphate buffer. According to some embodiments, the pharmaceutical composition comprises HEPES.

According to some embodiments, the composition is used as a coating on peri-implants. In other embodiments, the composition is administered in proximity to a peri-implant via injection to the soft tissue surrounding a peri-implant. In yet other embodiments, the composition is topically spread in an open crevice (for example a periodontal pocket) in proximity to a peri-implant.

The pharmaceutical compositions of the invention may be administered orally in various oral forms including, but not limited to, tablets, capsules, pills, powders, granules, elixirs, tinctures, suspensions, syrups, emulsions and as gel form. In instances in which oral administration is in the form of a tablet or capsule, the composition components can be combined with a non-toxic pharmaceutically acceptable inert carrier or excipients such as lactose, starch, sucrose, glucose, modified sugars, modified starches, methylcellulose and its derivatives, mannitol, sorbitol, and other reducing and non-reducing sugars, magnesium stearate, stearic acid, sodium stearyl fumarate, glyceryl behenate, amorphous silica gel or other desiccant material and the like.

According to certain embodiments, the pharmaceutical composition is in the form of a gel or salve. In this form, it is usually introduced into the gingival pockets, where it releases the active agent to the surroundings.

According to some embodiments, the pharmaceutical composition is in the form of a toothpaste.

According to other embodiments, the composition disclosed herein further comprises a thickening agent. In various embodiments, the thickening agent comprises at least one of hydroxypropyl methylcellulose, hydroxypropyl cellulose, hydroxyethyl cellulose, hydroxymethyl cellulose, carboxy methyl cellulose, polyvinylpyrrolidone, and polyvinyl alcohol. Each possibility represents a separate embodiment.

According to some embodiments, the composition disclosed herein further comprises an inorganic mineral. In several embodiments, the inorganic mineral comprises at least one of hydroxyapatite, calcium phosphate, calcium carbonate, calcium gluconate, calcium oxalate, calcium sulfate, calcium chloride, magnesium phosphate, magnesium carbonate, magnesium gluconate magnesium oxalate, magnesium sulfate, magnesium chloride, zinc phosphate, zinc carbonate, zinc gluconate, zinc oxalate, zinc sulfate, zinc chloride, and sodium bicarbonate. Each possibility represents a separate embodiment. In currently preferred embodiments, the inorganic mineral is a calcium phosphate mineral selected from the group consisting of amorphous calcium phosphate, tricalcium phosphate and hydroxyapatite. Each possibility represents a separate embodiment of the invention.

The pharmaceutical compositions described herein may typically be administered in daily doses of from about 1 μg to about 1 mg of the peptide described herein. According to certain embodiments, the pharmaceutical compositions described herein are administered in daily doses of from about 0.5 μg to about 500 μg of the peptide described herein. According to alternative embodiments, the pharmaceutical compositions described herein may typically be administered in daily doses of from about 5 μg to about 500 μg of the peptide described herein.

According to some embodiments, the peptide is administered in a daily dose of from 0.5 μg to 1 mg, 0.5 μg to 500 μg, 1 μg to 900 μg, 1 μg to 800 μg, 1 μg to 700 μg, 5 μg to 600 μg, 5 μg to 500 μg, 10 μg to 400 μg, 20 μg to 300 μg, or 50 μg to 250 μg. Each possibility represents a separate embodiment of the invention.

The pharmaceutical compositions or combinations described herein may typically be administered in daily doses of from about 0.5 mg to about 1000 mg CBD and 1 μg to about 1 mg of the peptide described herein. According to certain embodiments, the pharmaceutical compositions or combinations described herein are administered in daily doses of from about 0.5 mg to about 500 mg CBD and 0.5 μg to about 500 μg of the peptide described herein. According to alternative embodiments, the pharmaceutical compositions or combinations described herein may typically be administered in daily doses of from about 1 mg to about 300 mg CBD and 5 μg to about 500 μg of the peptide described herein.

According to some embodiments, the CBD is administered in a daily dose of from 0.1 mg to 1000 mg, 0.5 mg to 500 mg, 0.3 mg to 900 mg, 0.5 mg to 800 mg, 0.5 mg to 700 mg, 1 mg to 600 mg, 1 mg to 500 mg, 10 mg to 400 mg, 20 mg to 300 mg, or 50 mg to 250 mg. Each possibility represents a separate embodiment of the invention.

According to some embodiments, the peptide is administered in a daily dose of from 0.5 μg to 1 mg, 0.5 μg to 500 μg, 1 μg to 900 μg, 1 μg to 800 μg, 1 μg to 700 μg, 5 μg to 600 μg, 5 μg to 500 μg, 10 μg to 400 μg, 20 μg to 300 μg, or 50 μg to 250 μg. Each possibility represents a separate embodiment of the invention.

According to certain embodiments, the pharmaceutical composition is administered every two or three days. According to additional embodiments, the pharmaceutical composition is administered weekly. According to certain embodiments, the pharmaceutical composition is administered weekly for at least a months, two months, or three months.

As used herein the term “about” in reference to a numerical value stated herein is to be understood as the stated value +/−10%.

The following examples are presented in order to more fully illustrate some embodiments of the invention. They should, in no way be construed, however, as limiting the broad scope of the invention. One skilled in the art can readily devise many variations and modifications of the principles disclosed herein without departing from the scope of the invention.

### Examples

### Materials and Methods

Bone cell cultures—New-born mouse calvarial osteoblasts were prepared from 5 days-old mice by successive collagenase digestion (Smoum, Bar et al. 2010 Proc Natl Acad Sci USA 107: 17710-17715). Human osteoblasts were obtained from the cancellous bone of the head of the femur from patients undergoing total hip replacement (Helsinki ethics approval 0063-12-TLV) as reported previously (Gartland, Rumney et al. 2012 Methods Mol Biol 806: 337-355). For proliferation assays, cells pooled from 5-6 mice or from one patient were plated in 24-well format in triplicate, grown to subconfluence in α-MEM supplemented with 10% foetal calf serum (FCS) and then serum-starved for 2 h. Cell counts were determined after an additional 48-hour incubation in α-MEM supplemented with 0.5% BSA and the tested compound, OGP or HU910, with or without SR144528 (CB2 selective antagonist), at the indicated concentrations. Osteoclastogenic cultures were established from bone marrow-derived monocytes of 10-11-week-old mice and grown for 4 to 5 d in medium containing M-CSF (CMG[14-12] supernatant (Faccio, Takeshita et al. 2003 J Clin Invest 111: 749-758) and RANKL (R&D Systems) as reported previously (Asagiri and Takayanagi 2007 Bone 40: 251-264). Cells from 2 mice per genotype were pooled together and plated in 6 wells in a 96-well plate format. For osteoclast cell counts the osteoclastogenic cultures were fixed in formaldehyde and then stained for tartrate-resistant acid phosphatase (TRAP, Sigma) (Smoum, ibid). All in vitro experiments were replicated independently at least 3 times. The BRDU assay for the determination of osteoblast proliferation was performed as previously reported (Miguel, Namdar-Attar et al. 2005 J Biol Chem 280: 37495-37502).
Molecular dynamics (MD) computational details—The crystal structure of the CB2 receptor, recently reported, presents an inactive conformation (Li, Hua et al. 2019 Cell 176: 459-+), whereas that of CB1 receptor presents an active conformation (Hua, Vemuri et al. 2017 Nature 547: 468-+). The two receptors belong to the same sub-family (of lipid-cannabinoid receptors). The percent identity and similarity between the different segments of the CB1 and CB2 show that they share about 44% identity (62% similarity without the N- and C-terminal regions). A homology model of active CB2 was therefore constructed based on alignment to the structure of CB1. The Crystal structure and the homology model of CB2 are, thus, referred to as the inactive and active CB2 models, respectively. The active model was constructed based on alignment to the structure of CB1 (PDB ID: 5XRA) (Hua, Vemuri, ibid) after elimination of the fused Flavodoxin, using GPCRdb in Modeller 9.19 (Webb and Sali 2016 Current Protocols in Bioinformatics 54:5.6.1-5.6.37, 47:5.6.1-5.6.32, Pandy-Szekeres, Munk et al. 2018 Nucleic Acids Research 46: D440-D446). The inactive model involved replacing the fused T4-lysozyme in the CB2 crystal structure (PDB ID: 5ZTY) (Li, Hua, ibid) with ICL3, reverting mutations back into wild type residues and filling up the gaps in the structure. Docking calculations utilized Glide (Friesner, Banks et al. 2004 Journal of Medicinal Chemistry 47: 1739-1749) and MD simulations involved NAMD (Phillips, Braun et al. 2005 Journal of Computational Chemistry 26: 1781-1802). OPLS3 force field was applied in all docking calculations (Harder, Damm et al. 2016 Journal of Chemical Theory and Computation 12: 281-296). Protonation states of titratable residues were adjusted to pH=7.4. Two plausible binding sites within the model of the CB2 receptor were identified using the sitemap module of Schrödinger (Halgren 2009 Journal of Chemical Information and Modeling 49: 377-389). Ligand and peptide docking options of Glide were used to dock CP55,940 and 5aa-OGP, respectively. A cubical grid of 30 Å centered at the centroid of D25 and F183 was generated covering the two possible binding sites. The lowest energy conformations of peptide-receptor complex, which are expected to represent complexes with the highest binding propensity, were used to further comprehend the binding. The lowest energy conformation of 5aa-OGP bound to the inactive model of CB2 at the ECL region in the presence and absence of CP55,940 were subjected to MD simulations in NAMD (Philip Braun, ibid). Initial structure for MD has been prepared by www.charmm-gui.org web portal using charmm36 force field (Best, Zhu et al. 2012 Journal of Chemical Theory and Computation 8: 3257-3273). Homogeneous (1-palmitoyl-2-oleoyl-sn-glycero-3-phosphocholine) POPC lipid bilayer was built around the protein in a rectangular box. The protein-lipid complex was solvated using the TIP3P water model in an orthorhombic water-box with water height of ˜30 Å distance above and below the lipid bilayer. A restrained minimization was carried out for 10000 steps using the conjugate gradient method. The minimized system was then equilibrated for 100 ps while keeping protein and lipid molecules restrained followed by additional 600 ps gradually releasing these restraints. The temperature was set to 310.15K. Finally, the system was propagated for 100 ns using NPγT thermodynamic ensemble.
Effect of OGP on the ^{[35S]}GTPγS binding—Membranes prepared from CHO-KI cells expressing CB2 or control empty vectors (50 g per well) were incubated with tritiated CP55,940 and a range of concentrations of 5aa-OGP. The method for measuring agonist-stimulated ^{[35S]}GTPγS binding to cannabinoid CB2 receptors was adapted from Cascio et al. 2010 (Cascio, Gauson et al. 2010 Br J Pharmacol 159: 129-141). The assays were carried out with GTPγS binding buffer (50 mM Tris-HCl, 50 mM Tris-Base, 5 mM MgCl₂, 1 mM EDTA, 100 mM NaCl, 1 mM dithiothreitol, 0.1% BSA) in the presence of ^{[35S]}GTPγS and GDP, in a final volume of 500 ml. Binding was initiated by the addition of ^{[35S]}GTPγS to the wells. Nonspecific binding was measured in the presence of 30 mM GTPγS. The reaction was terminated by a rapid vacuum filtration method using Tris-binding buffer as described previously (Cascio, Gauson, ibid), and the radioactivity was quantified by liquid scintillation spectrometry. Agonists were stored at −20° C. as 10 mM stock solutions dissolved in distilled water or DMSO.
Rescue of OVX-induced bone loss—To test the skeletal activity of 5aa-OGP, 8-9 weeks old WT, CB2^{-/-} mice, on a C57Bl/6J background, were subjected to bilateral OVX. The animals were not further treated for the next 6 weeks to allow for a substantial amount of bone loss and the establishment of a new bone remodelling balance (Gavish, Bab et al. 1997 Biochemistry 36: 14883-14888). The animals were then sub-cutaneously injected once a day, for additional 6 weeks, with phosphate buffered saline (PBS, negative controls), or 10 ng/day of synthetic 5aa-OGP (test compound). The femora were then subjected to bone structural analyses by microcomputed tomography using a μCT system (μCT 50, Scanco Medical AG, Switzerland) as described below (Benito, Kim et al. 2005 J Neurosci 25: 2530-2536).
5aa-OGP prevention of age-related bone loss—Exogenous 5aa-OGP was administered to compensate for the decline in the endogenous levels of this hormone over time. To this end, three-month-old male mice were treated with 1 μg/kg of 5aa-OGP or PBS (control group), once daily 5 days a week for 3 more months. At the age of 6 months, mice were euthanized, and bones were harvested and analysed with μCT.
Structural skeletal micro computed tomography (μICT) analysis—Trabecular and cortical bone was examined in the femoral distal metaphysis and mid-diaphysis, respectively, as reported previously (Hiram-Bab, Liron et al. 2015 FASEB J 29: 1890-1900). Briefly, femora were collected, fixed in phosphate buffered formalin for 48 h and further kept in 70% ethanol. They were examined by a Scanco μCT50 (Scanco Medical AG, Switzerland) system. Scans were performed at an isotropic 10-μm nominal resolution, with 90 kVp energy, 114 mA intensity and 1100 msec integration time. The mineralised tissues were differentially segmented by a global thresholding procedure (140 and 224 permil for the trabecular and cortical bone, respectively). The bone parameters were determined using a direct 3D approach and data are presented in accordance with the official nomenclature (Bouxsein, Boyd et al. J Bone Miner Res 25: 1468-1486).
Age-related serum levels of OGP(1-14) in female subjects—Healthy female volunteers were selected after completing a detailed questionnaire related to their medical history. The exclusion criteria included the use of medications known to affect bone and mineral metabolism (other than oral contraceptives), and conditions such as diseases of the parathyroid, thyroid, kidney and liver. In addition, subjects who had experienced blood loss or bone fractures within the last 6 months were excluded, since these conditions may affect the serum level of OGP(1-14). Subjects were given specific instructions regarding sleep, diet and water consumption at 6 hours prior to the blood extraction. Subjects included 28 women aged 18-29 years, 6 women aged 30-39 years and 6 women aged 40-49 years. Blood samples, 5-10 ml in volume, were taken between 11:00 am and 12:30 pm. Samples were incubated at room temperature for 30 minutes, centrifuged for serum separation and kept frozen until testing.
OGP(1-14) determination via radioimmunoassay (RIA)—RIA to determine immunoreactive (ir)OGP(1-14) was carried out using rabbit anti-OGP(1-14) antibody raised against the C-terminal region of OGP(1-14), as described before (Gavish, ibid). This antiserum detects both the full-length OGP(1-14) and the truncated 5aa-OGP forms of the peptide. This assay is based on competitive binding to the antibodies of native and radio-iodinated peptide. The reaction mixture consisted of 50 μl of the test sample and 100 μl of each of the following solutions: 1:40 dilution of non-immune rabbit serum, 1:500 dilution of rabbit anti-OGP(1-14) antiserum and 4×10⁴ cpm of [3-¹²⁵I(Tyr¹⁰)] OGP(1-14). After overnight incubation at room temperature, the mixture was supplemented with 1 ml of a 1:50 diluted solution of goat anti-rabbit IgG (Sigma Chemical Co., St. Louis, MO, catalogue no. R0881) and 2.5% polyethylene glycol (Sigma Chemical, catalogue no. P2263). This was followed by additional 2 h shaking at 4° C. and centrifugation at 4,000×g for 15 min. The total radioactivity in the pellet was estimated in a gamma counter and irOGP(1-14) levels were calculated. The RIA intra- and inter-assay precision was approximately 5%.
Statistics—For a sample size greater than or equal to 6 and with normal distribution, differences between time/treatment groups were analyzed by Student's t-test (for 2 groups only) or ANOVA (for 3 groups or more). When significant differences were indicated by ANOVA, the group means were compared using the Fisher-LSD test for pairwise comparisons. Multiple group comparisons with n<6 were analyzed using non-parametric ANOVA. A p value less than 0.05 was considered statistically significant. For the human serum analysis, both the Kruskal-Willis one-way ANOVA and the Mann Whitney-Wilcoxon rank sum tests were used.
Study approval—Animals—C57BL/6J mice were used in all experiments. All procedures involving animals were carried out in accordance with the institutional guidelines and were approved by the Institutional Animal Care and Use Committee of Tel Aviv University (permit number M-14-092) and the Hebrew University of Jerusalem (permit number MD-12-13458-3). CB2 knockout (CB2^{-/-}) were generated and shipped from the University of Bonn (Germany) and bred in the respective animal facilities at the Hebrew University and Tel Aviv University (SPF unit).

Human osteoblasts—The cells were obtained from patients undergoing total hip replacement (Helsinki ethics approval 0063-12-TLV).

Human serum—The protocol was designed in accordance the institutional guidelines and with the approval of the Institutional Research Committee for Human Studies of the Hebrew University-Hadassah Medical Centre.

Calvarial Osteolysis Model. To test the direct effect of Ti particles on inflammation-induced osteolysis in vivo, a mouse calvaria model and μCT analysis specifically designed in our laboratory was used.

Membrane preparation—fibrin membranes, which were used as scaffolds to localize area of interest for treatment and analysis were prepared. Membraned made of fibrinogen from bovine plasma, mixed with thrombin from bovine plasma (Sigma-Aldrich, St. Louis, MO, USA) were made in 48-well plate either with embedded Ti particles originated from different Ti surface, with no particles as controls or with different treatments.

Surgical insertion model—After anesthesia, the skin of C57Bl/6J-Rcc female mice was shaved and disinfected at 10 weeks of age. The parietal bones of the mice were exposed via a 10 mm incision in the nape area, and the periosteum was removed using a periosteal elevator. Membranes were inserted to cover both parietal bones. The surgical incision was then closed using nylon monofilament surgical sutures (5/0). After a follow-up period of 5 weeks, the animals were sacrificed and the skull of each mouse was removed, fixed for 24 hrs in 4% phosphate-buffered formalin followed by ethanol 70%.

μCT analysis—All specimens were scanned and analyzed using a μCT system (CT 50, Scanco Medical AG, Switzerland). Scans were performed at a 10 m resolution in all three spatial dimensions, with 90 kV energy, 88 μA intensity and 1000 projections at a 1000 msec integration time. The region of interest (ROI) was defined as two 3.7 mm circles in the center of the parietal bones. The mineralized tissues were differentially segmented using a global thresholding procedure.

A custom-made algorithm based on Image-Processing Language (IPL, Scanco Medical) was developed to isolate the resorption pits, defined as unmineralized pits that were 10 to 40 m deep on the bone surface. The measured resorption volume was limited to a 40 m depth because beyond that, the resorption pit was connected to the internal diploe. Morphometric parameters were determined using a direct 3D approach (Bouxsein M L et al., Journal of Bone and Mineral Research. 2010; 25:1468-1486) and included the total volume of the bone resorption (Pit Resorption Volume, PRV, mm3) and bone tissue volume inside the ROI (TV, mm3), which was used to determine the PRV/TV (%).

### Example 1: CB2-5Aa-OGP Docking Followed by Molecular Dynamics (MD) Simulation

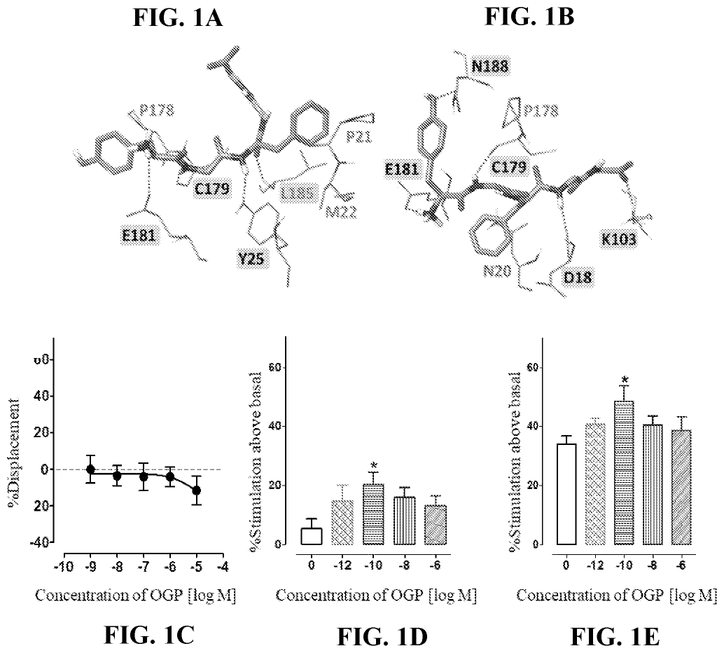

Docking studies of 5aa-OGP to both the active and inactive models of CB2 were used to predict feasible binding sites of 5aa-OGP in CB2. These studies did not rule out binding of 5aa-OGP at the orthosteric domain but suggested another potential binging site—possibly an allosteric site—at the extracellular region of CB2. The latter, while not being identical in the two receptor models, was very similar and involved in both cases mainly the N-terminal, ECL1 and ECL2 (Table 1). Furthermore, as one would expect from a receptor agonist, docking calculations suggest that 5aa-OGP exhibits higher affinity to the active model compared to the inactive model (Table 1). These studies also suggested that 5aa-OGP and CP55,940 bind to CB2 simultaneously, at distinct sites without reciprocally interfering. Indeed, MD simulations with 5aa-OGP at the proposed binding site within the ECL region show that the peptide stably stays in that region (FIG. 1A) with no noticeable changes in the dynamic energy even in the presence of the classical agonist CP55,940 (FIG. 1).

TABLE 1
Glide docking score (in kcal/mol) obtained for binding
5aa-OGP in active and inactive models of CB2

| | Inactive | Active |
|---|---|---|
| Binding complex | CB2_{X-ray} | CB2_{HM} |
| 5aa-OGP in ECL | −7.71 | −9.29 |
| 5aa-OGP in TM | −10.59 | −11.62 |
| 5aa-OGP in ECL in the presence of CP55,940 in | −7.09 | −9.35 |
| TM | | |

### Example 2: 5Aa-OGP Acts as a Positive Allosteric Modulator (PAM) in the Presence of a Lipophilic CB2 Agonist

Following the docking simulation that supported the notion of a stable binding of 5aa-OGP in the presence of a lipophilic agonist in the classical intramembranous binding site, a competition assay between tritiated CP55,940, a synthetic lipophilic CB2 agonist, and 5aa-OGP was performed. The results showed that 5aa-OGP did not displace CP55,940 suggesting that there is no competition between these agonists at the same binding site (FIG. 1C). In the absence of competition at the orthosteric binding site, it was next examined whether 5aa-OGP binding at an allosteric site modulates the functional activity of orthosteric agonists. hCB2-CHO-derived membranes were treated with CP55,940 and measured receptor activation using the GTPγS binding assay (FIGS. 1D, 1E). Notably, increasing concentrations of 5aa-OGP up to 10-10 M resulted in significantly higher CB2 activation by CP55,940 as indicated by the stimulation of ^{[35S]}GTPγS induced by 0.1 nM CP55,940 (FIG. 1D) and 10 nM CP55,940 (FIG. 1E). These results further support the notion that 5aa-OGP binds at an allosteric site at CB2 and indicate that 5aa-OGP acts as a PAM in the presence of other CB2 agonists.

### Example 3: CB2 is Essential for Maintaining the Proliferative Activity of 5Aa-OGP in Murine and Human Osteoblasts

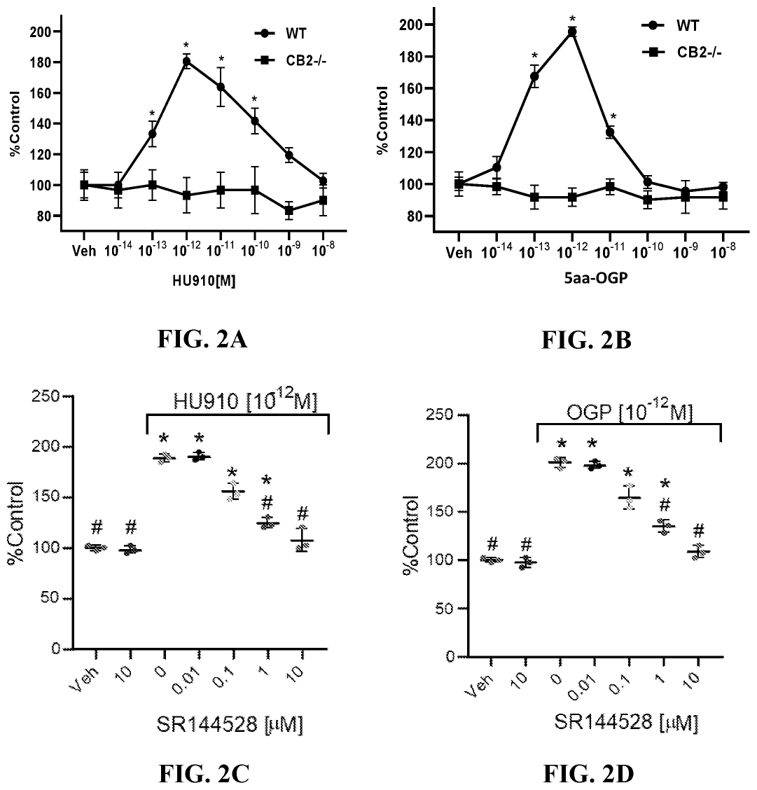

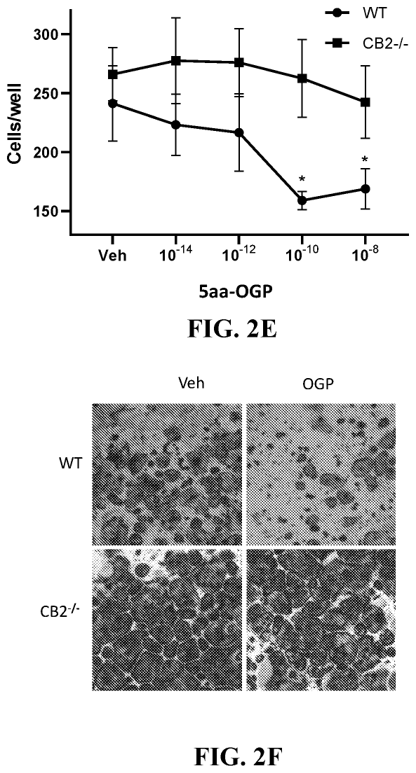

It is shown here in mouse-derived cultures that the osteoblastic proliferative activity of both 5aa-OGP and HU-910, a potent CB2-selective agonist, is characterized by nearly an identical dose response relationship (FIGS. 2A and 2B). The activity of 5aa-OGP is completely abrogated following genetic ablation of CB2 (FIG. 2B). The proliferative effect of 5aa-OGP in human osteoblasts (FIGS. 2B and 2C) was also abrogated following pharmacological blockade with the CB2-selective antagonist SR144528. These results indicate that the presence of CB2 is essential for 5aa-OGP activity in osteoblasts.

### Example 4: CB2 Mediates the OGP Attenuation of Osteoclast Differentiation

It is shown here that 5aa-OGP has anti-osteoclastogenic activity in cultures derived from WT animals (FIGS. 2E and 2F). This effect of 5aa-OGP is entirely absent in CB2^{-/-} derived cultures (FIGS. 2E and 2F), demonstrating that the expression of CB2 is essential for mediating the anti-osteoclastogenic effect of 5aa-OGP.

### Example 5: 5Aa-OGP Administration Rescues Sex Steroid Depletion-Related Bone Loss in Adult Female Mice in a CB2-Dependent Manner

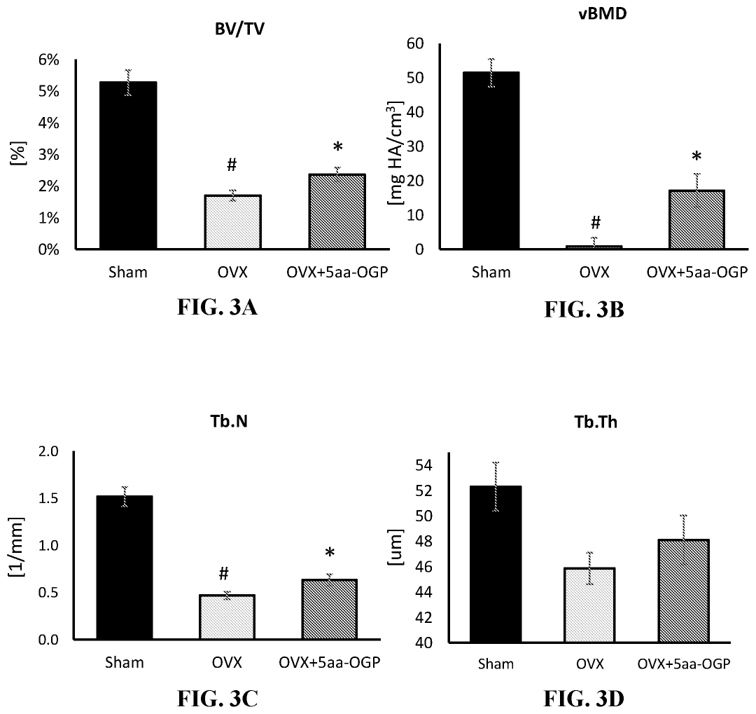

Here we employed the ovariectomy (OVX)-induced bone loss model where female mice were treated with OGP 6 weeks post-OVX. As expected, the vehicle-treated OVX animals (both WT and CB2^{-/-}) showed a significant bone loss in the trabecular bone compartment compared to the Sham-OVX control animals (FIGS. 3A-3C). Daily treatment of OVX mice with 5aa-OGP significantly restored the trabecular bone volume fraction (BV/TV) in the trabecular bone compartment (+45.9% vs. OVX, VEH-treated mice, p=0.035, FIG. 3A) of WT animals. This was accompanied with significant increases in trabecular volumetric bone mineral density (vBMD; FIG. 3B), Trabecular number (Tb.N; FIG. 3C), and Trabecular thickness (Tb.Th, FIG. 3D). 5aa-OGP did not affect any of the cortical bone parameters in the WT mice. Notably, 5aa-OGP had no effect on any of the measured bone parameters in OVX CB2^{-/-} animals (data not shown).

### Example 6: Age-Related Changes in Serum Levels of 5Aa-OGP and its Precursor OGP(1-14) in Humans

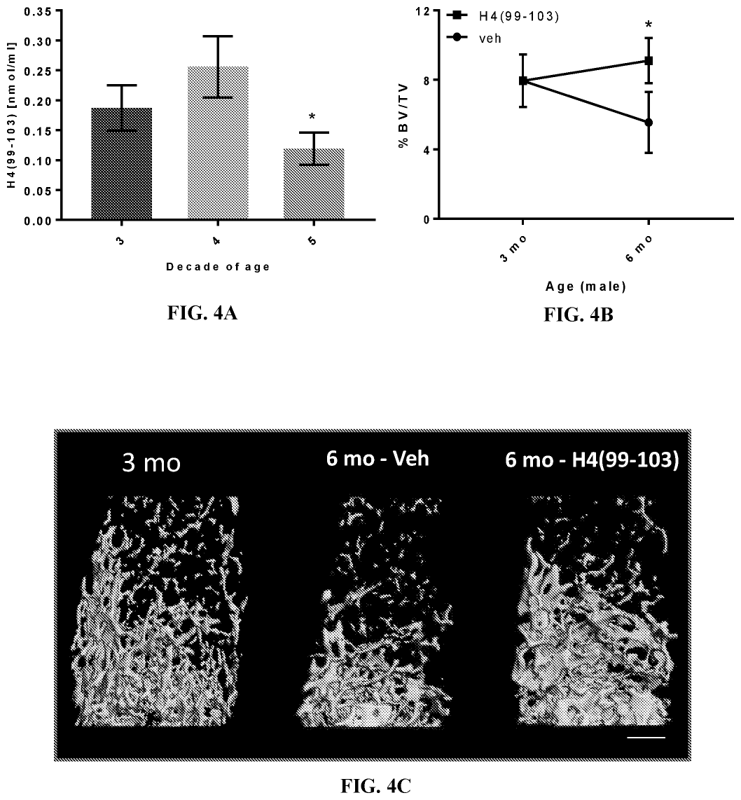

OGP(1-14), the precursor of 5aa-OGP, is secreted by stromal cells to the serum, where it binds to α2-macroglobulin. Upon its release, the 14-amino acid peptide is cleaved into the active pentapeptide. Both are detected by the same antibodies, hence the name immunoreactive (ir) OGP(1-14), which also includes the levels of free 5aa-OGP. In a previous study we reported that the ratio between free 5aa-OGP and total irOGP(1-14) in the serum decreased between the 3rd and the 5th decades of life in men. Importantly, this age range is characterized by age-related bone loss in humans, independently of sex hormones. Here, we investigated whether irOGP(1-14) serum levels decline with age during the same period in premenopausal women. Our analysis revealed that during the fifth decade of life irOGP(1-14) serum levels are significantly lower than during the 3rd and 4th decades (p=0.039 and p=0.016, respectively, FIG. 4A). This finding supports the notion that serum OGP(1-14) and 5aa-OGP levels decline with age, thus contributing to the reduced CB2 tone and related bone loss.

### Example 7: 5Aa-OGP Administration Completely Abrogates Age-Related Bone Loss in Adult Male Mice

Owing to the limited serum volume in mice, we were not able to accurately measure the serum levels of OGP(1-14)/5aa-OGP in mice. Nevertheless, to assess the role of 5aa-OGP in preventing age-related bone loss, we experimentally investigated the skeletal effect of exogenous administration of 5aa-OGP during adulthood. To this end, we treated 3-month-old male mice with 5aa-OGP or vehicle (VEH) for 3 months. This age range is characterized by slow age-related bone loss that follows the peak bone mass at 3 months of age. Indeed, VEH-treated mice (6mo-VEH) displayed a 30% trabecular bone loss relative to the 3-month-old mice (bone volume fraction, BV/TV, p=0.01, FIG. 4B). At this age the reduced BV/TV is associated with a significant decrease in trabecular connectivity density and number, with no change in trabecular thickness and in the cortical bone. On the other hand, daily administration of 1p g/kg/day OGP for 3 months completely prevented this bone loss (FIG. 4B), since the treated mice at 6 months of age displayed a BV/TV similar to their 3-month-old counterparts (p=0.124. The trabecular connectivity density, number and thickness were all significantly increased by 5aa-OGP (data not shown).

### Example 8: 5Aa-OGP Single Topical Administration Completely Abrogates Ti Particle-Induced Osteolysis In Vivo

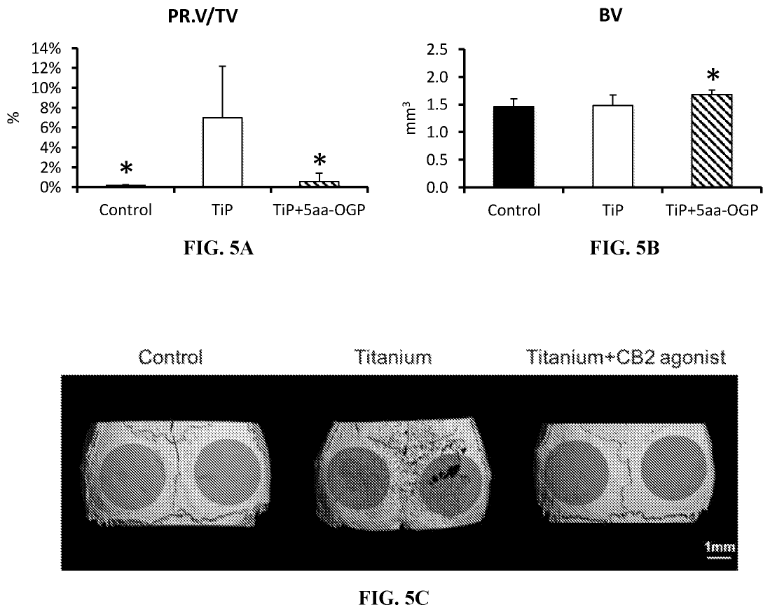

Using the same mouse model as previously published (Eger et al. Front Pharmacol. 2021; 12:638128 & Eger et al. Front Immunol. 2018; 9:2963), the efficacy of topical administration of 5aa-OGP in the prevention of inflammation-induced osteolysis was tested. Briefly, fibrin membranes including titanium particles derived from sand-blasted Ti implants, were also loaded with 10⁻¹¹M 5aa-OGP (TiP+5aa-OGP) or with saline (TiP) and implanted onto mice calvaria. Membranes with no TiP were inserted in the control group. Mice were sacrificed 4 weeks post-op. and subjected to μCT analysis. Our analysis revealed that the presence of Ti particles derived from sand-blasted implants induced a severe osteolysis (FIG. 5A). Topical administration of 5aa-OGP completely prevented this bone loss to levels similar to the unloaded membranes, despite the presence of Ti particles (FIG. 5A). In addition, 5aa-OGP had a bone anabolic effect resulting in bone thickening and increased bone volume in the calvaria (FIG. 5B). The 3D images clearly emphasize this dramatic effect of topical administration of 5aa-OGP (FIG. 5C).

### Example 9: Effect of the Combination of Pentapeptide 5Aa-OGP and CBD on Bone Repair

The therapeutic effect of a combination of the pentapeptide 5aa-OGP and CBD is assessed on bone regeneration in rat model of bone fracture as described in Gabet et al. (Bone 35 (2004); 65-73). The therapeutic effect is examined versus each active agent alone and versus placebo, and is further validated in a model of healing following orthopedic surgery in large animals (sheep). Each compound (or saline) is injected daily for 4 and 8 weeks. At sacrifice, the fractured bones are collected and analyzed using microCT (for structural and mineral density values) and biomechanical testing (resistance to re-fracturing).

Each compound is administered at 3 doses. A total of 7 groups of 15 animals is used. Starting dosages: Pentapeptide: 90, 270 and 900 ng/kg/day—CBD: 5, 15 and 50 mg/kg/day.

### Example 10: Enhanced Healing Following Tibial Tuberosity Advancement (TTA) Surgery

Tibial tuberosity advancement (TTA) surgery is a widely performed procedure in the treatment of ruptured cranial cruciate ligament in the knee articulation. This type of rupture is the most common knee injury and the most common orthopedic lameness in dogs.

This experiment is conducted in 50 kg sheep. The minimum number of animals in each group is 15 (45 animals total). A formula comprising the CBD+5aa-OGP peptide, or only CBD or 5aa-OGP is injected daily, 5 days a week for the entire duration of the experiment.

The recommended dosage for sheep and minipigs (above 40 kg of weight) is similar to that of humans. The regimen of CBD and 5aa-OGP is determined based on Example's 1 results.

Blood is collected from all animals on the day following surgery, and on a weekly basis for 10 weeks. Animals are monitored for weight, general well-being and infection/inflammation at the surgical site. In addition, mobility of the animals including weight bearing on the operated limb is scored by a single veterinarian on a scale from 1-6. After 10 weeks, tissues are harvested and analyzed. Measurable outcomes include:
Blood is collected once a week and kept for toxicology tests
Micro-CT evaluation of the bone tissue morphology along the section plan
Biomechanical testing of the tensile/shear force required to re-fracture the original section plan
Fourier-transform infrared spectroscopy (FTIR) analysis

In an in vitro experiment, osteoblasts are examined for proliferation and differentiation assays with each compound alone and combined together. In addition, collagen secretion and deposition are evaluated as well as 3D organization of the collagen fibrils and fibers.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

1-40. (canceled)

41. A method for treating or preventing a periodontal-related disease or condition comprising administering to a subject in need of such treatment, a therapeutically effective amount of a peptide comprising the amino acid sequence YGFGG (SEQ ID NO: 1; also denoted 5aa-OGP).

42. The method of claim 41, wherein the periodontal-related disease is periodontitis or peri-implantitis.

43. The method of claim 41, wherein the peptide consists of the sequence YGFGG, or wherein the peptide is osteogenic growth peptide (OGP) (SEQ ID NO: 2; also denoted OGP(1-14)).

44. The method of claim 43, wherein the peptide is administered in combination with an additional CB2 receptor agonist.

45. The method of claim 41, wherein the peptide is formulated in a pharmaceutical composition, further comprising an excipient, carrier or diluent.

46. The method of claim 45, wherein the method comprises administering the pharmaceutical composition at a daily dose comprising from about 0.5 to about 500 μg/day of the peptide.

47. The method of claim 45, wherein the method comprises administering the pharmaceutical composition at least once a week.

48. A pharmaceutical composition comprising: (1) a peptide comprising the amino acid sequence YGFGG (SEQ ID NO:1), and (2) CBD, a CBD derivative or an analog thereof, and a carrier, diluent or excipient, wherein the peptide is up to 50 amino acid residues in length.

49. The pharmaceutical composition of claim 48, wherein the peptide consists of the amino acid sequence YGFGG (SEQ ID NO:1; 5aa-OGP), or wherein the peptide is osteogenic growth peptide (OGP) (SEQ ID NO: 2).

50. The pharmaceutical composition of claim 48, comprising a dosage unit of between 0.5 and 500 mg CBD and between 0.5 and 500 μg peptide.

51. A method of treating a bone fracture comprising administering to a subject in need of such treatment a therapeutically effective amount of a pharmaceutical composition according to claim 48.

52. A method of treating a bone fracture comprising administering to a subject in need of such treatment a pharmaceutical combination of a therapeutically effective amount of a peptide comprising an amino acid sequence YGFGG (5aa-OGP; SEQ ID NO:1), and CBD or a derivative or analog thereof.

53. The method according to claim 52, wherein the treatment accelerates bone growth in a subject having a bone condition selected from the group consisting of a fracture by external force, pathological fracture, bone metastasis, fatigue fracture, distraction osteogenesis, osteotomy, osseointegration and combinations thereof.

54. The method according to claim 52, wherein the treatment comprises a distraction osteogenesis procedure and wherein the pharmaceutical combination is administered during the latency phase of the distraction osteogenesis procedure, the distraction phase of the distraction osteogenesis procedure, the consolidation phase of the distraction osteogenesis procedure or any combination thereof, or wherein the pharmaceutical combination is administered starting at least one day before a surgery that includes displacement or damage to a bone.

55. The method according to claim 52, wherein the method comprises accelerating bone consolidation following bone distraction.

56. The method according to claim 52, wherein the treatment comprises an osseointegration procedure, and wherein the pharmaceutical combination is administered following the implant insertion stage of the osseointegration procedure, following the abutment insertion stage of the osseointegration procedure or a combination thereof.

57. The method according to claim 56, wherein the osseointegration procedure comprises a dental implant integration.

58. The method according to claim 52, wherein the fracture is a fracture associated with osteoporosis, osteomalacia, malignant tumor, multiple myeloma, osteogenesis imperfecta congenita, cystic bone, suppurative myelitis, osteopetrosis, cancer-affected bone, or a combination thereof, or
wherein the bone fracture is triggered by an external force and results with a non-union fracture, mal-union fracture or delayed union fracture.

59. The method according to claim 52, wherein the pharmaceutical combination is administered at least once a day, and wherein the method comprises administering a pharmaceutical composition comprising the peptide at a daily dose comprising from about 0.5 to about 500 μg/day of the peptide, and administering a pharmaceutical composition comprising the CBD or combination at a daily dose comprising from about 0.5 to about 500 mg of the CBD, derivative or analog thereof.

60. The method according to claim 52, wherein the peptide and the CBD, CBD derivative or an analog thereof, are administered together or sequentially.

61. The method according to claim 52, wherein the peptide and the CBD, CBD derivative or an analog thereof are administered in the same pharmaceutical composition.

62. The method according to claim 52, comprising a step of directly administering of the peptide and/or CBD to the fracture site, or adjacent to the fracture site, or wherein the administering route is selected from the group consisting of orally, nasally, subcutaneously, intravenously, intramuscularly, intradermally, or via inhalation.
